(19) United States
(12) Patent Application Publication
(10) Pub. No.: US 2017/0000972 A1
(43) Pub. Date: Jan. 5, 2017

(54) COATED MEDICAL APPARATUS AND METHODS

(71) Applicant: Lenn R. HANN, Wheaton, IL (US)

(72) Inventor: Lenn R. HANN, Wheaton, IL (US)

(21) Appl. No.: 15/267,699

(22) Filed: Sep. 16, 2016

Related U.S. Application Data
(63) Continuation-in-part of application No. 14/312,362, filed on Jun. 23, 2014.
(60) Provisional application No. 62/220,364, filed on Sep. 18, 2015.
(60) Provisional application No. 62/266,585, filed on Dec. 12, 2015.
(60) Provisional application No. 61/941,889, filed on Feb. 19, 2014.

(51) Int. Cl. A61M 25/00; B05D 7/00; B05D 5/08; B05D 1/02

(52) U.S. Cl. CPC A61M 25/00; B05D 1/02; B05D 7/54; B05D 5/08; A61M 2025/0183

(57) ABSTRACT

A medical apparatus, such as a cannula tip for a peripheral vein of a human body, wherein the medical apparatus includes a micro- or nano-structured superhydrophilic basecoat and a liquid topcoat, together comprising a superhydrophobic coating, which inhibit occlusion and/or catheter related bloodstream infection. The topcoat can further include compatible drugs and/or biomaterials to enhance compatibility and/or enhance durability of the topcoat.

### CROSS REFERENCE TO RELATED APPLICATIONS

This application claims the benefit of both U.S. Provisional Application 62/220,364, filed on 18 Sep. 2015, and U.S. Provisional Application 62/266,585, filed on 12 Dec. 2015, and is also a continuation-in-part of U.S. patent application Ser. No. 14/312,362, filed on 23 Jun. 2014, which claims the benefit of U.S. Provisional Application 61/941,889, filed on 19 Feb. 2014. The co-pending parent applications are hereby incorporated by reference herein in its entirety and is made a part hereof, including but not limited to those portions which specifically appear hereinafter.

### BACKGROUND OF THE INVENTION

Field of the Invention

This invention relates to a coated apparatus and methods of use and manufacture, and more particularly, to an intravenous medical apparatus having a micro- or nano-structure basecoat and a liquid topcoat, together comprising a superhydrophobic coating.

Discussion of Related Art

Medical devices, including peripheral intravenous devices, are widely used for patients that require medication, fluids, and similar care applications found in hospitals and similar medical care facilities. Such devices include a cannula-over-needle apparatus, in which a flexible plastic or polymer cannula comes mounted on a metal insertion needle. Once the tip of the needle and cannula are located properly in the vein, the insertion needle is withdrawn and discarded. Meanwhile, the cannula is advanced inside the vein to a predetermined position where an external hub or valve area of the catheter is secured to the patient's body by medical tape or the like to hold it in place. Blood is often withdrawn at the time of the initial insertion of the cannula into the patient's vein to confirm placement. This is the most common intravenous access method used in both hospitals and in the field by paramedics or emergency medical technicians (EMTs).

The calibers of cannula generally range from 12 to 26 gauge with 12 being the largest and 26 being the smallest. The part of the catheter remaining outside of the skin is called the IV connecting hub or IV valve that is connected to the IV lines back to the IV bag of fluids. For example, an all-purposes IV cannula for infusions and blood draws might be an 18 and 20 gauge sized cannula manufactured by BD/Becton Dickinson or B. Braun. This intravenous cannula comes with an inner needle that is removed once the flexible portion of the cannula is fully inserted into the patient's vein.

Due to varying conditions within the medical facility and/or different skill levels of medical personnel inserting the IV device, such as a cannula, into the peripheral vein of a patient's hand or arm, complications sometimes develop in a number of the patients as a result. Such complications include occlusion which is a gradual blockage of the cannula which may result from improper device insertion, placement or the body's treatment of the cannula as a wound site. In addition, catheter related bloodstream infection (CRBSI), also referred to as catheter related sepsis, may result from catheters or similar devices that include the presence of bacteraemia. These conditions may arise as a result of improper insertion or occlusion of the cannula or as a result of contaminated devices or work areas. Many serious complications can result from sterilization issues, less-than-optimum cleaning, and/or improper cannula insertion into the vein. The potential complications include sepsis, edema causing tissue damage or may even include necrosis depending on the medication or fluid being infused. This extravasation is a leakage of infused fluids into the vasculature of the subcutaneous tissue surrounding the vein. The leakage of high osmotic solutions or chemotherapy fluids can result in significant tissue destruction or other complications.

### SUMMARY OF THE INVENTION

The invention provides a method of use and manufacture of an anti-thrombotic, anti-occlusion medical apparatus, for use in situations where coagulation, clotting, or biological adhesion can inhibit apparatus function. The invention provides a medial apparatus, such as a cannula with an insertion needle, including a micro- or nano-textured basecoat positioned over an exterior and/or interior of the apparatus, and a liquid topcoat positioned on the basecoat. The topcoat is adapted to inhibit one or more detrimental effects of use of the medical apparatus. The liquid topcoat can include, without limitation, organic or synthetic oils, such as vegetable oils or nut oils, any lipid or food additive regarded as safe by the FDA.

The invention further includes a medical apparatus with a surface adapted to be subcutaneously implemented. The surface, e.g., exterior and/or interior, can be formed of any suitable material, such as polytetrafluoroethylene, urethane, and/or silicone. A micro- or nano-textured basecoat is disposed over at least a portion of the surface, and desirably most if not all of at least the subcutaneous portion of the apparatus. A superhydrophobic liquid topcoat is positioned on the basecoat; the superhydrophobic liquid topcoat inhibiting biological deposition on the surface, and thereby inhibiting, for example, occlusion and apparatus-related bloodstream infection.

The basecoat can be any suitable hydrophilic material. In some embodiments, the basecoat is a micro- or nano-textured superhydrophilic basecoat positioned over all or part of an external or internal surface of the apparatus. Examples of hydrophilic and/or superhydrophilic base coat materials include, without limitation, organic or synthetic waxes, starches, such as from corn or other vegetables, fiber, either soluble or insoluble plant fiber, plant source cellulose, crystallizing compounds, binding agents, or combinations thereof. The liquid topcoat is positioned on the basecoat, and they together forming a superhydrophobic coating that, for example, inhibits occlusion and apparatus related bloodstream infection.

Additional embodiments may include modifying compounds to assist in compatibility, medicine delivery and/or other benefits. Examples of suitable modifying compounds include chemical compounds, drugs, biocompatible compounds (e.g., lipids), or organism cells or fluids. The modifying compounds can be suitably matched to the intended patient, and may even be taken from the patient, processed, and combined with the topcoat, such as on-site, prior to the procedure to ensure compatibility.

The invention further includes methods of making and/or using the apparatus of this invention. In some embodiments, the method of manufacturing a medical apparatus includes: forming or providing the medical apparatus surface of or with at least one of polytetrafluoroethylene, urethane and silicone; coating the surface with a micro- or nano-textured basecoat; and coating the basecoat with a liquid topcoat positioned on the basecoat, together forming a superhydrophobic coating. The applied topcoat can be a modified liquid topcoat, such as modified with an integrated biocomponent, such as a fluid or cell, from the patient's body.

The above summary of the present invention is not intended to describe each illustrated embodiment or every implementation of the present invention. The figures and the detailed description which follow more particularly exemplify these embodiments.

### BRIEF DESCRIPTION OF THE DRAWINGS

This invention is explained in greater detail below in view of exemplary embodiments shown in the drawings, wherein:

FIG. 1 shows a schematic of a cannula in accordance with one embodiment of the present invention;

FIG. 2 shows a schematic of a cannula in accordance with one preferred embodiment of the present invention;

FIG. 3 shows a schematic of a cannula in accordance with one preferred embodiment of the present invention; and

FIG. 4 shows a schematic of a cannula in accordance with one preferred embodiment of the present invention.

### DESCRIPTION OF PREFERRED EMBODIMENTS

The present invention relates to an apparatus, such as for subcutaneous or intravenous contact or use with a patient, and methods for manufacturing and use of the apparatus.

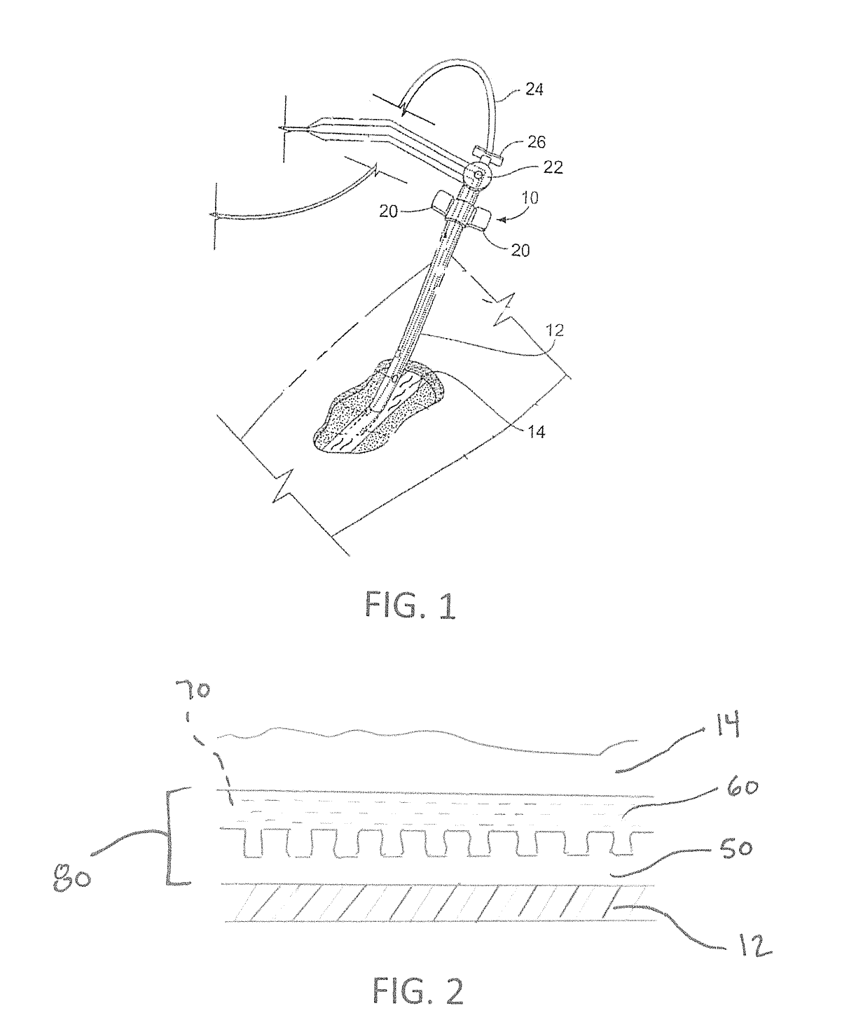

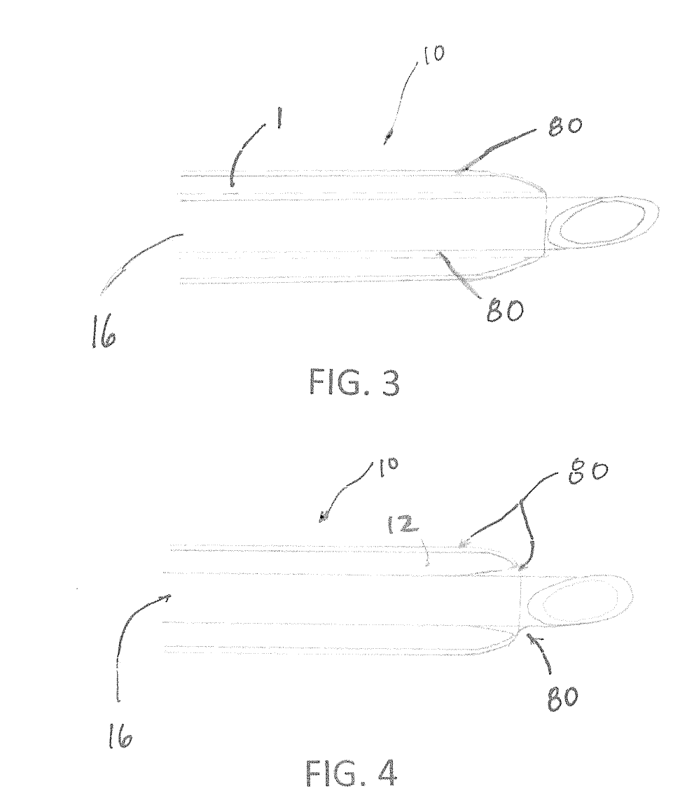

FIG. 1 illustrates an exemplary medical apparatus according to this invention, namely a catheter 10 constructed in accordance with the present invention. The typical catheter 10 includes a short polymer tube (a few centimeters long) inserted through the skin into a peripheral vein 14 (any vein generally not inside the chest or abdomen). This is usually in the form of a flexible cannula 12 over-needle device, in which a flexible plastic cannula 12 comes mounted on a metal insertion needle (insertion needle 16 not shown in FIG. 1 as already withdrawn from cannula 12, see FIGS. 3 and 4). Once the tip of the needle and cannula 12 are located within the vein 14 the insertion needle is withdrawn and discarded. The cannula 12 is further advanced inside the vein to an appropriate position and then secured with medical tape or the like over a pair of plastic wings 20 secured to the tubing near a port or hub 22. An IV line 24 may further connect to the port or hub 22 through a male fluid input 26 that is inserted into the IV line 24.

According to one preferred embodiment of this invention, the cannula 12 as shown and described includes a superhydrophobic coating and/or construction. Accordingly, the cannula 12 includes at least one of: a micro- or nano-coating over and/or within the cannula 12; the cannula 12 may be constructed of urethane, polytetrafluoroethylene or silicone; and/or a hybrid construction, such as oil impregnated urethane.

FIG. 2 shows a schematic of one embodiment of the invention wherein the cannula 12, such as a urethane cannula, includes a micro or nano-textured basecoat 50, over which a liquid topcoat 60 is applied. The basecoat 50 and liquid topcoat 60 are together referred to as the superhydrophobic coating 80. “Superhydrophobic” may be quantified as a coating wherein the contact angles of a water droplet exceed 150° and the roll-off angle/contact angle hysteresis is less than 10°. As described herein, the liquid topcoat 60 may be further modified to include one or more modifying compounds.

According to one preferred embodiment of this invention, a medical device such as an IV cannula, such as shown and described in U.S. Ser. No. 14/168,902, may include a superhydrophobic coating and/or construction. Accordingly, the cannula may include at least one of: a micro- or nano-structure basecoat 50 over and/or within the cannula 12; a superhydrophobic topcoat 60 over the basecoat; the cannula 12 may be constructed of urethane, PTFE (e.g., Teflon®) or silicone; and/or a hybrid construction, such as oil impregnated urethane.

A suitable basecoat 50 and/or topcoat 60 may comprise a coating such as described in U.S. Pat. Nos. 8,574,704 and 8,535,779 to Smith et al., which are hereby incorporated by reference. The Smith et al. patents describe non-wetting surfaces that include a liquid impregnated within a matrix of micro/nano-engineered features on the surface, or a liquid filling pores or other tiny wells on the surface. Such a product, called Liquiglide™, may be used to coat the cannula described herein. As described, a micro/nano-engineered surface coating enables a durable liquid-impregnated surface coating to be placed over the full exterior and interior surfaces of an IV cannula.

A benefit of such liquid-impregnated surface coating constructions is to inhibit the initial “seed” adhesion of blood protein fibrin to the cannula surface, thus preventing further fibrin accretion at the orifice of the tip of the cannula, thus preventing IV cannula occlusion.

The subject invention may be preferably utilized in connection with micro- or nano-scale coatings, such as described in U.S. Pat. Nos. 8,574,704 and 8,535,779 to Smith et al., to include application to several additional medical devices as follows: (1) peripheral IV cannulas 12, as described above; (2) central venous catheters (CVC); (3) peripherally inserted central catheters (“PICC”); (4) midline catheters and/or (5) subcutaneous cannulas used with wearable insulin and chemotherapy pumps. In such devices, the superhydrophobic coating is preferably applied to the cannula to prevent occlusion or catheter related bloodstream infections (CRBSIs).

The subject invention may be further or alternatively utilized in hemodialysis fistulas, specifically prosthetic hemodialysis access arteriovenous grafts (AVGs). In this application, the superhydrophobic topcoat 60 is preferably applied on cannula tips and within the fistula to prevent clotting.

The subject invention may be further or alternatively utilized in surgical drains used to evacuate body fluids generated during post-surgical wound healing. In this application, the superhydrophobic topcoat 60 is preferably applied at the tip of the drain and within to prevent clotting.

The subject invention may be further or alternatively utilized in stents used in vascular surgery to prevent blood coagulation, as well as other implanted stents that may benefit from a non-wetting superhydrophobic topcoat 60. Such stents include ureteral, urethral, biliary, duodenal, colonic, and pancreatic stents. In these applications, the superhydrophobic topcoat 60 is preferably applied over the entire area of the stent to prevent clotting, tissue adhesion, and other fluid adhesions.

Each of the described medical devices is subject to unwanted blood coagulation during normal use and operation. Significant savings in cost, infection risk, and patient discomfort can be made by adding micro- or nano-structure superhydrophilic basecoats 50 and/or superhydrophobic topcoats 60 to these devices.

According to one preferred embodiment, IV cannula coating clearance may be accommodated. The application of a liquid-containing superhydrophobic topcoat 60 to an IV cannula 12 preferably accounts for the thickness of the basecoat 50 and/or the topcoat 60 and utilizes a thinner cannula 12, a thinner hollow needle, and/or a larger diameter cannula 12 so that there is room for the interior basecoat 50 and/or topcoat 60.

According to one preferred embodiment, the urethane cannula surface is prepared for optimum basecoat 50 adhesion. Methods of doing this may include: plasma ion treatment, heat and vacuum or some combination of these three.

Further, the shelf life of one or more components of the subject invention may be of concern. For instance, the lifespan of a superhydrophobic liquid topcoat 60 once applied to a urethane catheter 12, then sterilized and packaged should be accommodated. Basecoats 50 and/or superhydrophobic topcoats 60 according to this invention preferably utilize FDA approved compounds to build their coatings, including starches and waxes, including beeswax, for the basecoat 50, and water, food-grade oils, including mineral, palm and organic oils, and silicone or synthetic oils for the superhydrophobic topcoat 60. A preferred combination permits FDA approval in human IV use while also providing acceptable shelf life prior to use. A starch/beeswax basecoat 50 and a organic or synthetic oil top coat 60 should provide an adequate balance between FDA approval and acceptable shelf life. The assembly can also be shipped with liquid for recoating or initial coating prior to use.

An additional solution to improve shelf life according to one embodiment of the invention is wet storage. In this embodiment, the cannula assembly may be stored in a liquid-filled package. The liquid would preferably be identical to the liquid top coat 60 of the superhydrophobic coating 80 of the cannula 12. Such storage method would inhibit liquid loss due to evaporation, osmosis or other packaging porosity effects. As mentioned above, the package can also be shipped with the topcoat liquid for initial coating or recoating prior to use.

According to one preferred embodiment, a coated cannula 12 should be sterilized for packaging. As described above, such packaging should preferably have suitable shelf life for potentially years of storage prior to use. Preferred methods for this task include ionizing radiation, either gamma ray or electron beam. Alternative, or in addition, gas treatment, either ethylene oxide or formaldehyde may be utilized in connection with improving shelf life. However, this method must include safeguards against gas impingement or absorption into the liquid surface coat. Alternatively, or in addition, autoclave heat treatment may be used provided it does not damage the structure of any FDA-approved starch/wax basecoat. Alternatively, or in addition, an aseptic assembly and packaging may be utilized.

In some embodiments of the present invention the superhydrophobic topcoat 60 includes one or more modifying compounds 70 thereon or therein. Exemplary modifying compounds include at least one of chemical compounds, drugs, biocompatible compounds, organism cells or fluids, and/or other substances for conveyance into the vein for the duration of IV use. The nature of the topcoat and the modifying compound concentration can be adjusted to determine any desired release rate.

In one example, the superhydrophobic topcoat 60 may include a modifying compound 70 comprising an amount of the drug Alteplase (Cathflo Activase) to aid in preventing catheter occlusion from fibrin adhesion. Another preferred drug for this purpose may be Drotrecogin alfa (Xigris) which also aids in preventing sepsis. In another example, the liquid topcoat 60 may contain a modifying compound 70 comprising antibiotic, anti-sepsis or anti-inflammatory drugs, or any combination thereof.

In embodiments of the present invention, a specific makeup of superhydrophobic topcoat 60 is tailored for specific applications. Specifically, one objective is to increase biocompatibility between the medical device (e.g., stent, portacath, or any long-team implanted device) and the human host. To accomplish this, the topcoat 60 is created or augmented using components of compatible blood or bodily fluid, such as the patient's own blood. The most likely candidates to improve biocompatibility are the patient's plasma and the patient's platelet rich plasma (PRP), which is extracted after a centrifuge process. A topcoat 60 including the patient's blood components will increase biocompatibility and reduce inflammation, clotting, and immune responses. This custom-tailored liquid top coat may be further combined with one or more other modifying compounds as described above to carry drugs or other modifying compounds. An organic or synthetic lipid or other oily topcoat 60 may be preferable for applications incorporating a modifying compound, such as a body fluid (e.g., plasma, PRP) liquid topcoat. PRP may contain a number of biological growth and healing factors, such as platelet-derived growth factor; transforming growth factor beta; fibroblast growth factor; insulin-like growth factor 1 or 2; vascular endothelial growth factor; epidermal growth factor; Interleukin 8; keratinocyte growth factor; and/or connective tissue growth factor.

Other modifying compounds, such as for improving biocompatibility, include, without limitation, stem cells, such as adipose tissue stein cells, bone marrow cells, and other patient donor biologic materials. In addition, benefits may be found in using bovine and/or porcine submaxillary mucin and/or human sublingual mucin as a component of the subject top coats along with various other human or patient donor cells. Other various human and animal epithelial and/or other cells can also improve biocompatibility.

According to one preferred embodiment of the invention, the basecoat 50 may utilize a hardened beeswax to create a “self-healing” property to the superhydrophobic topcoat 60. One objective of this embodiment is to engineer a basecoat 50 that resists internal body degradation and seeks to bind with body fluids. This would of course prove advantageous for an implanted medical device and could lead to greater biocompatibility and tissue integration. This embodiment is further useful for a stent or IVG used for dialysis.

As described above, a preferred method of manufacture of the subject medical device includes forming a surface from at least one of polytetrafluoroethylene, urethane and/or silicone; coating the surface with a micro- or nano-textured basecoat 50; and coating the basecoat 50 with a liquid topcoat 60 positioned on the basecoat 50, the basecoat 50 and topcoat 60 together forming a superhydrophobic coating 80.

In addition, multiple topcoat formulations may be utilized depending on where the coating occurs. For instance, a first topcoat can be positioned within an interior of the apparatus and a second topcoat, having different properties from the first topcoat, can be positioned on an exterior of cannula 12.

Conventional assembly techniques for urethane IV catheter and insertion needle are established and inexpensive. However, as described above, such techniques suffer the significant problems of occlusion and catheter related blood stream infections (CRBSIs). One objective of the present invention is to eliminate occlusion and CRBSIs through the use of superhydrophobic coatings, including superhydrophobic coatings modified to contain chemicals, drugs, body fluids and modified body fluids.

The subject invention can be manufactured using one of several methods. For instance, for a catheter, full-length internal coating, is possible where clearance between the metal insertion needle 16 (as shown in FIGS. 3 and 4) and the catheter 10 is feasible. In such a method, a full-length coating (of one or both of the basecoat 50 and the topcoat 60) can be applied to the interior of the cannula 12. This coating may differ between the external (blood stream contact) coating and the internal (metal insertion needle to urethane cannula) coating. The internal coating might be engineered to resist sticking, friction and compression. The internal coating may be further engineered to resist shearing off or other damage during assembly with the metal insertion needle. Such internal coating may be a higher specific gravity based formulation.

Another possible method of manufacture involves partial, orifice only internal coating. The cannula 12 in this method may be designed to include a flared orifice at the tip, such as shown in FIG. 4. Accordingly, the insertion needle 16 may be assembled in a conventional manner and the exterior of the cannula 12 may be coated as well as the interior of the flared portion of the cannula. In this manner, superhydrophobic coatings are placed where needed to prevent occlusion/CRBSIs and leave the remainder of the cannula interior uncoated as per current practice. In this embodiment, the coating can be sprayed on after insertion needle 16 is assembled into the catheter 10. This can leave a superhydrophobic coating “fillet” at the orifice that may be useful in preventing occlusion. Once the insertion needle is withdrawn, the liquid top coat fillet may retract to form a liquid torus shape at the orifice of the catheter.

Also note that the infusate might become mixed or partially mixed with the liquid topcoat 60 of the superhydrophobic coating 80. In such event, the liquid topcoat may be applied to minimize mixing with the infusate.

Another embodiment of this invention addresses problems related to damage to the superhydrophobic coating 80 that manifest when the cannula 12 is inserted through the skin of the patient. The coating 80 may be compressed, thinned or sheared during passage through the skin or vein. To mitigate such risk, one solution is to engineer the liquid topcoat 60 to resist the damage through the addition of human compatible gelling or thickening agents to the liquid topcoat 60. Such gelling agents permit the coating 80 to retain superhydrophobic properties but would be toughened to increase insertion durability and overall reliability during the term of use within the patient.

Additional embodiments include medical applications for using the above described coatings to coat the exterior and interior of: PEG (percutaneous endoscopic gastrostomy) feeding tubes, gastric feeding tubes; NG (nasogastric) feeding tubes; NJ (Nasojejunal) feeding tubes; GJ (gastrojejunostomy) feeding tubes; J-tube (jejunostomy) feeding tubes; and gastric drainage tubes and other enteral feeding and drainage tubes. According to one preferred embodiment, the apparatus is a synthetic and/or tissue-based graft or mesh (similar to stents) that can be coated with a base coat as described and a tailored top coats to improve biocompatibility. Additional applications can be found in percutaneous endoscopic gastrostomy; urinary catheters; nasogastric intubation; and enteral administration.

The aforementioned base coats and top coats can be improved with improved methods of application. Such improved methods can include vacuum deposition to the I.D. of a tube for several (1-5) cm during exterior coating; vacuum application using dipping into a reservoir, vacuum and expulsion using air to dry the coating; and/or electrostatic application using spray and/or vacuum and/or dipping.

A manufactured, non-liquid, base coat can utilize methods other than spraying the standard chemical mixture. The goal is to produce a nano- and micro-textured surface that may be complex in texture, possibly fractal. One example is a 3D snowflake shape crystalline structure. Methods to produce this surface can include vacuum crystal deposition, vacuum polymer deposition, laser or plasma surface etching, various forms of masking then etching, and/or surface crystallization.

According to one preferred embodiment of the invention, sterile portable aerosol medical top and base coatings are used for surgical applications. Different applicators and/or containers can contain different top coats and different base coats and can be applied in various combinations depending on the procedure. Applications include but are not limited to: coating tissues to prevent surgical adhesions; coating tissues to aid in wound drainage; coating tissues to reduce bacterial infection; coating tissues to reduce friction and inflammation; coating tissues to reduce edema; and/or coating implanted devices, meshes, sutures, staples, attachment points, etc. to reduce all of the above: clotting, adhesions, bacterial infection, friction, edema, etc. One challenge in this variant will be spraying the base coat onto wet tissue and devices and having it adhere and/or cure properly. A drying agent or desiccant built into the base coat spray can aid in effectiveness, as can a catalyst to trigger/improve local attachment of the base coat.

The invention is useful for non-medical applications as well. Thickening and/or gelling agents can be used in the liquid top coat to increase the durability of the coating when used with abrasive products. For example, the invention, with or without a modifying compound, can be used in containers for food or other products. As a specific container example, peanut or other nut butter (which are thicker and more abrasive) containers can be treated with the subject top coats and base coast to improve removal from the container.

One problem that may reduce the effectiveness of the subject invention relates to liquid top coat depletion. For full hydrophobicity, the liquid is preferably immiscible with blood and at a lower viscosity than blood, which is normally 40/100 mP (millipoise) at peak systolic velocity. Blood measures at much higher viscosity at low shear rates and is a non-Newtonian fluid. Water measures at 10/100 mP at any velocity.

In embodiments of this invention, for example, a blood-immiscible lipid (oily, greasy, fatty) liquid, solid or semi-solid at internal body temperature, and at a correspondingly higher viscosity than blood, would still show significantly greater inertness than PTFE coatings and would be depleted from the catheter at a much lower rate than a liquid with a lower viscosity than blood. This high degree of inertness could be hydrophobic enough to repel blood adhesion to the catheter. In such circumstances, use of such additive formulation of a medical grade coating could last for months on a PICC or midline catheter, and potentially longer.

Additionally or alternatively, a replenishing approach is used if the liquid top coat is made of the same lipids or other material used in venous feeding solutions. Venous total parenteral nutrition (TPN) feeding solutions contain a mix of different nutritional lipids along with proteins and carbohydrates. Using these lipids as the liquid coating of a midline or PICC catheter and in the feeding solution can replenish the liquid coating of the catheter as the lipid emulsion circulates through the bloodstream. Under such circumstances, the superhydrophobicity of the catheter could be sustainable indefinitely.

The process of infusing the feeding solution would re-coat the interior of the catheter, and the solution in the bloodstream would pass over the exterior of the PICC or midline catheter, and the natural affinity of the oil in the bloodstream for the base coat on the catheter would cause it to bond with the catheter, thus replenishing the liquid top coat. The replenishing topcoat liquid may also be supplied and/or infused separately, and not as a component of the TPN or other solution.

A coating according to the subject invention may be used in connection with balloon angioplasty, and other balloon expansion applications. This may provide usefulness in reducing friction with the artery or vein wall and also as a carrier for drugs to treat the expanded tissue as the balloon is activated.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

What is claimed is:

1. A medical apparatus comprising:
a micro- or nano-textured basecoat positioned over at least one of an exterior and interior of the apparatus; and
a liquid topcoat positioned on the basecoat, such topcoat inhibiting one or more detrimental effects of use of the medical apparatus.

2. The medical apparatus of claim 1 further comprising a superhydrophobic liquid topcoat that inhibits occlusion and related bloodstream infection.

3. The medical apparatus of claim 1 further comprising a superhydrophilic basecoat.

4. The medical apparatus of claim 1 wherein the exterior and interior of the apparatus is constructed of at least one of polytetrafluoroethylene, urethane, or silicone.

5. The medical apparatus of claim 1 further comprising a pretreated surface for coating adhesion, the pretreated surface comprising treatment with at least one of plasma ion treatment, heat and vacuum.

6. The medical apparatus of claim 1 further comprising a modifying compound within the liquid topcoat, wherein the modifying compound comprises at least one of chemical compounds, drugs, biocompatible compounds, or organism cells or fluids.

7. The medical apparatus of claim 6 wherein the modifying compound comprises a component of a solution for delivery into the injection site during use of the medical apparatus.

8. The medical apparatus of claim 6 wherein the modifying compound comprises a composition including a blood component, a body fluid, or a biological component from or compatible with a patient that will receive the medical apparatus.

9. The medical apparatus of claim 6 wherein the modifying compound comprises a lipid or a submaxillary or sublingual mucin.

10. The medical apparatus of claim 1, wherein the medical apparatus is selected from a catheter or cannula, a stent, a feeding tube, a drainage tube, or a synthetic or tissue-based graft or mesh.

11. The medical apparatus of claim 1, wherein the base coat and/or the top coat is portable and adapted to applied on-site during the use of the medical apparatus.

12. The medical apparatus of claim 1 wherein the liquid topcoat comprises at least one of organic or synthetic oil, saline, glycol, polyvinyl alcohol, glycine, or a human compatible gelling or thickening agent.

13. A medical apparatus, comprising:
a surface adapted to be subcutaneously implemented;
a micro- or nano-textured basecoat disposed over at least a portion of the surface; and
a superhydrophobic liquid topcoat positioned on the basecoat, such superhydrophobic liquid topcoat inhibiting biological deposition on the surface, thereby inhibiting occlusion and apparatus-related bloodstream infection.

14. The medical apparatus of claim 13 further comprising a superhydrophilic basecoat.

15. The medical apparatus of claim 14, wherein the basecoat comprises an organic or synthetic wax, a starch, soluble or insoluble fiber, plant source cellulose, crystallizing compounds, binding agents, or combinations thereof.

16. The medical apparatus of claim 15 wherein the liquid topcoat comprises at least one of organic or synthetic oil, saline, glycol, polyvinyl alcohol, glycine, or a human compatible gelling or thickening agent.

17. The medical apparatus of claim 16, wherein the base coat and/or the top coat is portable and adapted to applied on-site during the use of the medical apparatus.

18. A method of manufacturing a medical apparatus according to claim 13, comprising:
forming or providing the medical apparatus surface including at least one of polytetrafluoroethylene, urethane and silicone;
coating the surface with a micro- or nano-textured basecoat; and
coating the basecoat with a liquid topcoat positioned on the basecoat, together forming a superhydrophobic coating.

19. The method of manufacturing of claim 18, further comprising:
applying a modified liquid topcoat over the basecoat.

20. The method of manufacturing of claim 17, further comprising:
integrating a patient's body fluid into the modified liquid topcoat.
